(19) United States
(12) Patent Application Publication
(10) Pub. No.: US 2025/0063864 A1
(43) Pub. Date: Feb. 20, 2025

(54) LED LIGHT SOURCE FOR MEDICAL OPTICAL COHERENCE TOMOGRAPHY WITH HIGH AXIAL RESOLUTION

(71) Applicant: LUMILEDS LLC, San Jose, CA (US)

(72) Inventor: Peter Josef SCHMIDT, Aachen (DE)

(73) Assignee: LUMILEDS LLC, San Jose, CA (US)

(21) Appl. No.: 18/937,834

(22) Filed: Nov. 5, 2024

Related U.S. Application Data
(63) Continuation of application No. PCT/US2023/025735, filed on Jun. 20, 2023.
(60) Provisional application No. 63/355,837, filed on Jun. 27, 2022.

(51) Int. Cl. H01L 33/50; C09K 11/68; G01B 9/02091; H01L 33/32

(52) U.S. Cl. CPC H01L 33/502; C09K 11/685; G01B 9/02091; H01L 33/32

(57) ABSTRACT

This specification discloses spinel-type phosphors that may be advantageously employed in light sources for medical optical coherence tomography (OCT), light sources for medical OCT comprising such phosphors, and OCT systems comprising such light sources.

### CROSS-REFERENCE TO RELATED APPLICATIONS

This application is a continuation of PCT Application PCT/US2023/025735 filed Jun. 20, 2023, which claims benefit of priority to U.S. Provisional Patent Application 63/355,837 filed Jun. 27, 2022. Both of the above applications are incorporated herein by reference in their entirety.

### FIELD OF THE INVENTION

The invention relates generally to LED and pcLEDs.

### BACKGROUND

Semiconductor light emitting diodes and laser diodes (collectively referred to herein as “LEDs”) are among the most efficient light sources currently available. The emission spectrum of an LED typically exhibits a single narrow peak at a wavelength determined by the structure of the device and by the composition of the semiconductor materials from which it is constructed. By suitable choice of device structure and material system, LEDs may be designed to operate at ultraviolet, visible, or infrared wavelengths.

LEDs may be combined with one or more wavelength converting materials (generally referred to herein as “phosphors”) that absorb light emitted by the LED and in response emit light of a longer wavelength. For such phosphor-converted LEDs (“pcLEDs”), the fraction of the light emitted by the LED that is absorbed by the phosphors depends on the amount of phosphor material in the optical path of the light emitted by the LED, for example on the concentration of phosphor material in a phosphor layer disposed on or around the LED and the thickness of the layer.

Phosphor-converted LEDs may be designed so that all of the light emitted by the LED is absorbed by one or more phosphors, in which case the emission from the pcLED is entirely from the phosphors. In such cases the phosphor may be selected, for example, to emit light in a narrow spectral region that is not efficiently generated directly by an LED. Alternatively, pcLEDs may be designed so that only a portion of the light emitted by the LED is absorbed by the phosphors, in which case the emission from the pcLED is a mixture of light emitted by the LED and light emitted by the phosphors. By suitable choice of LED, phosphors, and phosphor composition, such a pcLED may be designed to emit, for example, white light having a desired color temperature and desired color-rendering properties

For biomedical imaging methods such as optical coherence tomography (OCT) broad band emitting high intensity light sources for the medical optical window spectral range (˜800-1300 nm) are required. Superluminescent diodes (SLDs) applied in commercial systems emit in the desired wavelength range but show significant limitations due to high cost, limited lifetime, low axial optical resolution (>10 μm typical) and low thermal stability of emission.

### SUMMARY

This specification discloses pcLEDs that may outperform SLDs in all aspects: low cost, high stability of emission with drive and temperature, and high reliability. These pcLED may have emission peak positions in the 1200 nm wavelength region which leads to a low effective attenuation coefficient in blood perfused biological tissue and low light scattering. The emission peaks may have a full width at half maximum (FWHM) which leads to high OCT axial resolutions of 3-4 μm, for example. Moreover, short wavelength infrared (SWIR) emission flux levels that exceed those of SLDs may be obtained.

The pcLEDs disclosed herein comprise a semiconductor LED and a wavelength converting structure comprising a spinel-type phosphor having the general formula AE_{1-x-z}A_{z+0.5(x-y)}D_{2+0.5(x-y)-z-u}E_{z}O₄:Ni_{y},Crᵤ, with AE=Mg, Zn, Co, Be or mixtures from this group of divalent atoms, A=Li, Na, Cu, Ag or mixtures from this group of monovalent atoms, D=Ga, Al, B, In, Sc or mixtures from this group of trivalent atoms and E=Si, Ge, Sn, Ti, Zr, Hf or mixtures from this group of tetravalent atoms; where 0≤x≤1, 0<y≤0.1, 0≤z≤1, 0≤u≤0.2.

A preferred phosphor system is defined by x=1, z=0, A=Li, D=Ga, Al: that is, Li_{0.5-0.5y}D_{2.5-0.5y-u}O₄:Ni_{y},Crᵤ with 0<y≤0.1, 0≤u≤0.2.

These spinel type phosphors have peak emission in the preferred range of 1100-1300 nm for biological tissue OCT with spectral widths (FWHM) in the 180-200 nm or 180-230 nm or 170-240 nm range enabling axial imaging resolutions in the 3-3.2 μm range. Infrared output power in the 1000-1700 nm range from pcLEDs comprising these phosphors may exceed 60 mW or exceed 100 mW when driven by 1 Watt of electric power delivered to a blue emitting LED.

Wavelength converting structures as disclosed herein may comprise the spinel-type phosphors disclosed herein in powder form dispersed in a binder such as a silicone binder, for example. Alternatively, wavelength converting structures as disclosed herein may comprise the spinel-type phosphors disclosed herein in ceramic form.

The pcLEDs disclosed herein may optionally comprise a phosphor that absorbs light emitted by the semiconductor LED and emits light absorbed by the spinel-type phosphor to provide the desired SWIR emission. In the absence of such an additional phosphor, the (e.g., blue) semiconductor LED emission directly excites emission from the spinel-type phosphor to provide the desired SWIR emission.

This specification also discloses light sources for OCT, OCT systems, and OCT methods employing the pcLEDs disclosed herein.

### BRIEF DESCRIPTION OF THE DRAWINGS

FIG. 1A illustrates an example OCT system.

FIG. 1B illustrates an embodiment of a wavelength converting structure as part of an illumination device.

FIG. 2 illustrates another embodiment of a wavelength converting structure as part of an illumination device.

FIG. 3 is a cross sectional view of an LED.

FIG. 4 is a cross sectional view of a device with a wavelength converting structure in direct contact with an LED.

FIG. 5 is a cross sectional view of a device with a wavelength converting structure in close proximity to an LED.

FIG. 6 is a cross sectional view of a device with a wavelength converting structure spaced apart from an LED.

FIG. 7 shows the X ray powder diffraction (XRD) pattern (Cu k alpha radiation) of the obtained Li_{0.532}Ga_{2.35}Sc_{0.1}O₄:Ni_{0.051}.

FIG. 8 shows the powder reflectance and emission (620 nm excitation) spectra of the obtained Li_{0.532}Ga_{2.35}Sc_{0.1}O₄:Ni_{0.051}.

FIG. 9 shows the X ray powder pattern (Cu k alpha radiation) of the obtained Li_{0.536}Al_{1.01}Ga_{1.44}O₄:Ni_{0.051}.

FIG. 10 shows the powder reflectance and emission (620 nm excitation) spectra of the obtained Li_{0.536}Al_{1.01}Ga_{1.44}O₄:Ni_{0.051}.

FIG. 11 shows the X ray powder pattern (Cu k alpha radiation) of the obtained Li_{0.273}Mg_{0.5}Sc_{0.05}Ga_{2.16}O₄:Ni_{0.05}.

FIG. 12 shows the powder reflectance and emission (620 nm excitation) spectra of the obtained Li_{0.273}Mg_{0.5}Sc_{0.05}Ga_{2.1604}:Ni_{0.05}.

FIG. 13 shows the X ray powder pattern (Cu k alpha radiation) of the obtained Li_{0.49}Sc_{0.05}Ga_{2.384}O₄:Ni_{0.013},Cr_{0.05}.

FIG. 14 shows the powder reflectance spectrum and emission spectrum (for 620 nm excitation) of the obtained Li_{0.49}Sc_{0.05}Ga_{2.384}O₄:Ni_{0.013},Cr_{0.05}.

FIG. 15 shows the thermal quenching behavior of emission under 620 nm excitation of the obtained Li_{0.49}Sc_{0.05}Ga_{2.384}O₄:Ni_{0.013},Cr_{0.05}.

FIG. 16 shows the X ray powder pattern (Cu k alpha radiation) of the obtained Li_{0.49}Al_{0.05}Ga_{2.384}O₄:Ni_{0.013},Cr_{0.05}.

FIG. 17 shows the powder reflectance spectrum and emission spectrum (for 620 nm excitation) of the obtained Li_{0.49}Al_{0.05}Ga_{2.384}O₄:Ni_{0.013},Cr_{0.05}.

FIG. 18 shows the emission spectrum of the phosphor converted LED of Example 8 for the visible (left) and IR (right) spectral range.

FIG. 19 shows the total emission spectrum of the pcLED of Example 7.

FIG. 20 shows the total emission spectrum of the pcLED of Example 8.

FIG. 21 shows an SEM micrograph of the surface of Example 9.

FIG. 22 shows the reflectance spectrum of Example 9.

FIG. 23 shows the emission spectrum of the pcLED of Example 10.

### DETAILED DESCRIPTION

As summarized above, this specification discloses spinel-type phosphors that may be advantageously employed in light sources for medical OCT. OCT is an interferometric optical imaging method that may be used to create three-dimensional images of tissue. OCT may be performed in the frequency domain or in the time domain. A detailed review of OCT can be found at https://www.ncbi.nlm.nih.gov/pmc/articles/PMC5418377/.

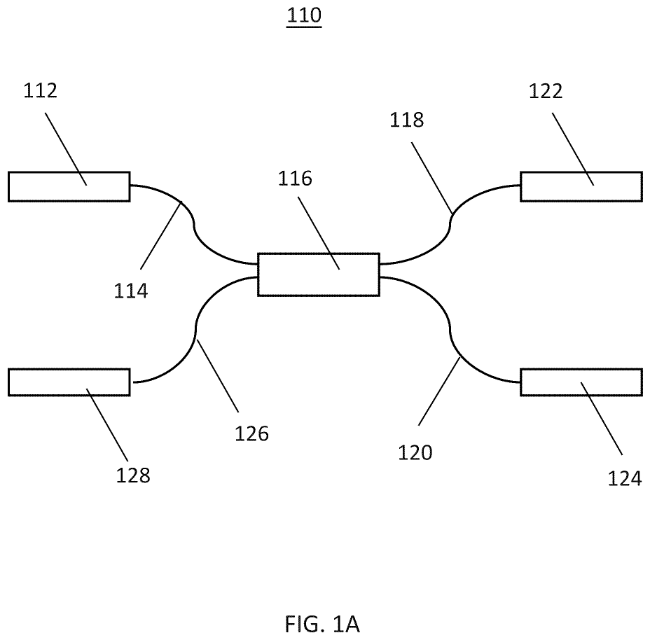

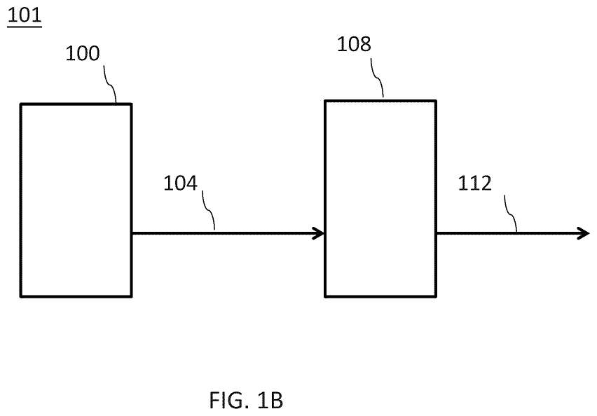

Referring to FIG. 1A, an example OCT system 110 is configured as an interferometer comprising a low coherence (i.e. broad band) light source 112 that provides light through optical fiber 114, optical coupler/splitter 116, and optical fibers 118 and 120 to a sample arm 122 and to a reference arm 124. Light transmitted into the sample arm is scattered by a sample in the sample arm back through optical fiber 118, optical coupler/splitter 116, and optical fiber 126 to a detector 128. Light transmitted into the reference arm is retroreflected by a moving or stationary reflector back through optical fiber 120, optical coupler/splitter 116, and optical fiber 126 to the detector 128. The retroreflected light from the reference arm and the back scattered light from the sample arm interfere at the detector, which generates a corresponding electric signal. The interference signal from the detector provides information about the structure of the sample from which light was backscattered.

The characteristics of the light source in the OCT system are an important design parameter because the axial resolution, also known as the depth resolution or the coherence gate, is the coherence length of the light source, an intrinsic parameter of the light source which is inversely proportional to its spectral bandwidth. When the spectral distribution of the light source is Gaussian, the axial resolution Δz is given as: Δz=2 ln 2/p*l₀²/FWHM where λ₀ and FWHM are the light source central wavelength and its bandwidth. Therefore, broadband optical sources are required in order to achieve high axial resolution. Axial resolutions in the micron and sub-micron range can be achieved by using sources with very large spectral bandwidth.

Alternatively, frequency-domain OCT systems may employ wavelength tunable light sources such as for example tunable lasers or broad band light sources used with a tunable wavelength selecting element.

The wavelength range of a light source suitable for OCT should be positioned within the “optical window” in the near infrared (NIR) range of the spectrum because light in the ˜700-1300 nm range has a long penetration depth in biological tissue. Oxyhaemoglobin in blood strongly absorbs at shorter wavelengths and water shows strong absorption at larger wavelengths. Especially for blood perfused biological tissue such as skin the wavelength range 1100-1300 nm is suitable, also because the scattering coefficient significantly drops at larger wavelengths.

This specification provides broad band light sources for OCT that alleviate the disadvantages of state-of-the art light sources. The disclosed OCT LED light sources may for example show axial resolutions in the 2.9-3.8 μm range for emission peak positions in the 1200-1250 nm range and spectral half widths FWHM in the 170-240 nm range.

A particular advantage of the disclosed OCT LED light sources is that they allow for the first time the construction of cost effective and even portable OCT systems due to their compact size and low cost drive electronics needed. The disclosed light sources are suitable for time and frequency domain OCT systems

### Compositions of Spinel Type SWIR Phosphors

Spinels have the general formula XY₂O₄, where X is a divalent cation and Y is a trivalent cation, for example, MgAl₂O₄, and crystallize in the cubic (isometric) crystal system. Normal spinel structures are cubic close-packed oxides with eight tetrahedral and four octahedral sites per formula unit. The tetrahedral spaces are smaller than the octahedral spaces. Y³⁺ ions occupy half the octahedral holes, while X²⁺ ions occupy one-eighth of the tetrahedral holes. Inverse spinel structures have a different cation distribution in that all of the X²⁺ ions and half of the Y³⁺ ions occupy octahedral sites, while the other half of the Y³⁺ ions occupy tetrahedral sites. Most spinel structures are somewhere between the normal and inverse spinel structure.

Compositions of spinels may have X=AE (“AE” is alkaline earth metal) with a charge of +2, and a Y cation with a charge of +3. However, the spinel structure also forms if X is a cation with charge +1 and Y is a cation with charge +4.

Compositions of spinel type SWIR phosphors that emit light in the 1000-1700 nm range may be AE_{1-x-z}A_{z+0.5(x-y)}D_{2+0.5(x-y)-z-u}E_{z}O₄:Ni_{y},Crᵤ with AE=Mg, Zn, Co, Be or mixtures from this group of divalent atoms, A=Li, Na, Cu, Ag or mixtures from this group of monovalent atoms, D=Ga, Al, B, In, Sc or mixtures from this group of trivalent atoms and E Si, Ge, Sn, Ti, Zr, Hf or mixtures from this group of tetravalent atoms; where 0≤x≤1, 0<y≤0.1, 0≤z≤1, 0≤u≤0.2.

Compositions of spinel type SWIR phosphors may have X=1 and z=0 (i.e., no AE and no E atoms) and A=Li and D=Ga and Al, Sc, or a mixture of Al and Sc, with Ga being the main component of D (i.e., having a higher concentration than Al and/or Sc). A preferred phosphor system for OCT light sources is defined by x=1, z=0, A=Li, D=Ga, Al: Li_{0.5-0.5y}D_{2.5-0.5y-u}O₄:Ni_{y},Crᵤ with 0<y≤0.1, 0≤u≤0.2.

Compositions of SWIR phosphors may have A atoms and D atoms such as Li and Ga replaced by AE atoms such as Mg (A+D replaced by 2 AE to maintain charge neutrality), which the inventors have found leads to a broadening of the emission band and increased emission intensity at lower energies. Substitution of part of the A atoms, such as Li, with AE atoms like Mg can be done without a significant change of properties (x<1). Zn can reduce the optical band gap and the luminescence efficiency. Co has about the same size as Mg, Zn or Li and also shows IR emission. The E atom may be Ge because it is known to form inverse spinel structures with e.g. Li and Ga also present.

Examples of spinel type SWIR phosphors are the inverse spinel phosphors Li_{0.475}AlGa_{1.475}O₄:Ni_{0.05} and Li_{0.225}Mg_{0.5}Ga_{2.225}O₄:Ni_{0.05} with the D cations Ga and Al occupying both the tetrahedral and octahedral sites of the spinel structure. The emission of these phosphors is located in the 1100-1600 nm wavelength range. The absorption and emission bands of these phosphors can be tuned by varying the average size of D cations. The inventors found that D cations larger than Ga, such as In or Sc, lead to a spectroscopic red shift, while cations smaller than Ga, such as Al or B, lead to a spectroscopic blue shift of absorption and emission bands.

Codoping of these inverse spinel SWIR phosphors with Cr³⁺ allows efficient excitation in the blue and red spectral range. The Cr³⁺ ions absorb most of the excitation energy and transfer it to the Ni²⁺ atoms that show broad band luminescence in the 1100-1600 nm wavelength range. An example for such phosphor composition is Li_{0.49}Sc_{0.05}Ga_{2.384}O₄:Ni_{0.013},Cr_{0.05}.

In the method for synthesizing the spinel type SWIR phosphor compositions, a flux additive may be used. Flux additives for the inverse spinel phosphors herein may be borate compounds such as lithium tetraborate, Li₂B₄O₇. When such a flux additive is used, small amounts of boron may be present in the SWIR phosphors after full processing, and such boron may improve the luminescence intenisites.

### Light Sources Including the Spinel-Type SWIR Phosphors

FIG. 1B illustrates a wavelength converting structure 108 that includes at least one of the disclosed luminescent SWIR spinel-type phosphors. Wavelength converting structure 108 is used in an illumination device 101, which includes primary light source 100. Illumination device 101 may be used as the light source for an OCT system as described above. Within device 101, the primary light source 100 may be an LED or any other suitable source including, as examples, resonant cavity light emitting diodes (RCLEDs) and vertical cavity laser diodes (VCSELs). Light source 100 emits a first light 104. A portion of the first light 104 is incident upon a wavelength converting structure 108. The wavelength converting structure 108 absorbs the first light 104 and emits second light 112. The wavelength converting structure 108 may be structured such that little or no first light is part of the final emission spectrum from the device, though this is not required.

Primary light source 100 may emit blue light and may be or comprise an InGaN LED, for example. Alternatively, primary light source 100 may emit longer wavelength light such as red light, for example, and may be or comprise an AlInGaP LED.

The wavelength converting structure 108 may include an SWIR phosphor that can be excited in the red spectral range. In such case, device 101 may include as a primary light source 100 a red emitting LED, emitting first light 104 in the 600-1000 nm wavelength range. In some embodiments, light source 100 is an AlInGaP type emitter and may emit first light 104 having wavelengths in the 600-650 nm range. In other embodiments, light source 100 may be an InGaAs type emitter and may emit first light in the 700-1000 nm wavelength range, to excite the disclose Ni²⁺ doped SWIR phosphors directly via the energetically lowest lying absorption transition.

In an example, device 101 may have a wavelength converting structure 108 that includes Li_{0.5-0.5x}(Ga,Sc)_{2.5-0.5x-y}O₄:Niₓ,Cr_{y} (where 0≤x≤1, 0<y≤0.1, 0≤z≤1, 0≤u≤0.2) spinel type SWIR phosphor, and may use as primary light source 100 a 620-630 nm emitting AlInGaP type LED. More specifically, device 101 may have a wavelength converting structure 108 that includes Li_{0.49}Sc_{0.05}Ga_{2.384}O₄:Ni_{0.013},Cr_{0.05} spinel type SWIR phosphor, and may use as primary light source 100 a 622 nm emitting AlInGaP type LED.

The wavelength converting structure 108 described with respect to FIG. 1 can be manufactured, for example, in powder form, in ceramic form, or in any other suitable form. The wavelength converting structure 108 may be formed into one or more structures that are formed separately from and can be handled separately from the light source, such as a prefabricated glass or ceramic tile, or may be formed into a structure that is formed in situ with the light source, such as a conformal or other coating formed on or above the source.

In some embodiments, the wavelength converting structure 108 may comprise powders that are dispersed for example in a transparent matrix, a glass matrix, a ceramic matrix, or any other suitable material or structure. SWIR phosphor dispersed in a matrix may be, for example, singulated or formed into a tile that is disposed over a light source. The glass matrix may be for example a low melting glass with a softening point below 1000° C., or any other suitable glass or other transparent material. The ceramic matrix material can be for example a fluoride salt such as CaF₂ or any other suitable material.

The wavelength converting structure 108 may be used in powder form, for example by mixing the powder SWIR phosphor with a transparent material such as silicone and dispensing or otherwise disposing it in a path of light. In powder form, the average particle size (for example, particle diameter) of the SWIR phosphors may be at least 1 μm in some embodiments, no more than 50 μm in some embodiments, at least 5 μm in some embodiments, and no more than 20 μm in some embodiments. Individual SWIR phosphor particles, or powder SWIR phosphor layers, may be coated with one or more materials such as a silicate, a phosphate, and/or one or more oxides in some embodiments, for example to improve absorption and luminescence properties and/or to increase the material's functional lifetime.

In powder form the phosphors may be mixed for example with curable phenyl methyl polysiloxane resins or dimethyl polysiloxane resins to form a phosphor in silicone suspension that may be cast into films to form converter parts after film curing and cutting or that may be directly dispensed into LED packages equipped with, for example, InGaN LED dies emitting in the 420-450 nm wavelength range. The powder phosphor may also be used to coat at least a part of a fiber optical element that is optically coupled to a pump light source to form a OCT light source.

Alternatively and even more preferred, the disclosed spinel type phosphors are first fired to obtain reactive precursor powders that are then sintered into polycrystalline ceramics. A forming option may be ceramic tape casting followed by tape drying, cutting, stacking, lamination, de-bindering and sintering to form free-standing ceramic wafers that can be diced into platelets to attach them onto, for example, InGaN type LED dies emitting in the 420-450 nm wavelength range.

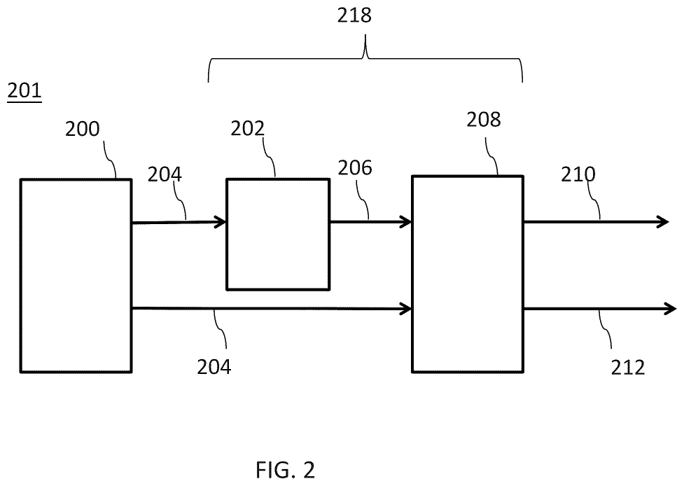

FIG. 2 illustrates another embodiment in which a wavelength converting structure including one or more of the disclosed SWIR phosphor materials may further be combined with a second phosphor system. In FIG. 2, the wavelength converting structure 218 includes an SWIR phosphor portion 208 and a second phosphor portion 202 as part of an illumination device 201. Illumination device 201 may be used as the light source for an OCT system as described above.

In FIG. 2, a light source 200 may be an LED or any other suitable source, (including as examples resonant cavity light emitting diodes (RCLEDs) and vertical cavity laser diodes (VCSELs). Light source 200 emits first light 204. First light 204 is incident upon wavelength converting structure 218, which includes an SWIR phosphor portion 208 including one or more of the SWIR phosphors disclosed herein, and a second phosphor system 202. A portion of the first light 204 is incident on a second phosphor portion 202 of the wavelength converting structure 218. The second phosphor 202 absorbs the first light 204 and emits third light 206. The third light 206 may have a wavelength range that is within the excitation range of the SWIR phosphor in the SWIR phosphor portion 208 of the wavelength converting structure 218. The third light 206 is incident on the SWIR phosphor portion 208. The SWIR phosphor portion 208 absorbs all or a portion of the third light 206 and emits fourth light 210. Additionally, a portion of the first light 204 may be incident on an SWIR phosphor portion 208 of the wavelength converting structure 218. The SWIR phosphor portion 208 may absorb the first light 204 and emit second light 212, or first light 204 may pass through the SWIR phosphor portion 208.

The wavelength converting structure 218 including an SWIR phosphor 208 and second phosphor 202 may be structured such that little or no first light or third light is part of the final emission spectrum from the device, though this is not required.

The SWIR phosphor portion 208 of wavelength converting structure 218 may include, for example, one or more of the Ni2+, or Ni2+ and Cr2+ spinel phosphors disclosed herein, such that device 201 emits light in the 1000-1700 nm wavelength range.

Any suitable second phosphor may be used in the second phosphor system 202. The second phosphor system 202 can be used with SWIR phosphor portion 208 to widen the spectral range that allows efficient excitation of the SWIR phosphor, and thus increase number of the types of primary light sources 200 that may be used in device 201. That is, the second phosphor system 202 may include a second phosphor that absorbs first light 204 from a primary light source 200 that emits light outside of the wavelength range required to excite the SWIR phosphor. For instance, the second phosphor system 202 may absorb first light 204 emitted from a blue or green LED as primary light source 200. The second phosphor system 202 then emits third light 206 in the red spectral range. The third light 206 emitted from second phosphor system 202 excites the SWIR phosphor in SWIR phosphor portion 208.

For example, device 201 may include green to red emitting phosphors, such as Eu2+ phosphors, added as the second phosphor system 202, and may use a blue emitting LED as the primary light source 200. Examples of a red emitting phosphor for use in second phosphor system 202 include (Sr,Ca)AlSiN₃:Eu and (Ba,Sr,Ca)₂Si₅₋ₓAlₓOₓN₈₋ₓ:Eu.

In an example device, primary light source 200 may be a blue light emitting InGaN type emitter. The wavelength converting structure 218 may include an orange-red emitting (Ba,Sr)₂Si₅N₈:Eu phosphor as the second phosphor system 202 and a Li_{0.5-0.5x}(Ga,Sc)_{2.5-0.5x-y}O₄:Niₓ,Cr_{y} spinel phosphor as the SWIR phosphor portion 212. In particular, device 201 may include as a primary light source 200 a 440-455 nm emitting InGaN type emitter, and a wavelength converting structure 218 that includes orange-red emitting phosphor (Ba_{0.4}Sr_{0.6})₂₋ₓSi₅N₈:Eu_{0.02} and a Li_{0.49}Sc_{0.05}Ga_{2.384}O₄:Ni_{0.013},Cr_{0.05} SWIR phosphor.

The second phosphor system 202 may include Cr³⁺ doped phosphors that emit in the 700-1000 nm wavelength range and that can be excited in the blue to green and red spectral ranges. The emission light of such Cr3+ phosphors being reabsorbed by Ni²⁺ doped SWIR phosphor disclosed herein.

The second phosphor system 202 may include other Ni²⁺ phosphor systems that are known from the literature. Examples include LaMgGa₁₁O₁₉:Ni, MgO:Ni, MgF₂:Ni, Ga₂O₃:Ni,Ge, or garnets of composition RE₂AEMg₂TV₃O₁₂:Ni (RE=Y, La, Lu, Gd, Nd, Yb, Tm, Er; AE=Ca, Sr; TV=Si, Ge).

The wavelength converting structure 218 including SWIR phosphor 208 and the second phosphor 202 described with respect to FIG. 2 can be manufactured, for example, in powder form, in ceramic form, or in any other suitable form. The SWIR phosphor 208 and the second phosphor 202 may be formed into one or more structures that are formed separately from and can be handled separately from the light source, such as a prefabricated glass or ceramic tile, or may be formed into a structure that is formed in situ with the light source, such as a conformal or other coating formed on or above the source.

The SWIR phosphor 208 and the second phosphor 202 may be mixed together in a single wavelength converting layer, or formed as separate wavelength converting layers. In a wavelength converting structure with separate wavelength converting layers, SWIR phosphor 208 and the second phosphor 202 may be stacked such that the second phosphor 202 may be disposed between the SWIR phosphor 208 and the light source, or the SWIR phosphor 208 may be disposed between the second phosphor 202 and the light source.

In some embodiments, the SWIR phosphor 208 and the second phosphor 202 may be powders that are dispersed for example in a transparent matrix, a glass matrix, a ceramic matrix, or any other suitable material or structure. Phosphor dispersed in a matrix may be, for example, singulated or formed into a tile that is disposed over a light source. The glass matrix may be for example a low melting glass with a softening point below 1000° C., or any other suitable glass or other transparent material. The ceramic matrix material can be for example a fluoride salt such as CaF₂ or any other suitable material.

The SWIR phosphor 208 and second phosphor 202 may be used in powder form, for example by mixing the powder phosphor with a transparent material such as silicone and dispensing or otherwise disposing it in a path of light. In powder form, the average particle size (for example, particle diameter) of the phosphors may be at least 1 μm in some embodiments, no more than 50 μm in some embodiments, at least 5 μm in some embodiments, and no more than 20 μm in some embodiments. Individual phosphor particles, or powder phosphor layers, may be coated with one or more materials such as a silicate, a phosphate, and/or one or more oxides in some embodiments, for example to improve absorption and luminescence properties and/or to increase the material's functional lifetime.

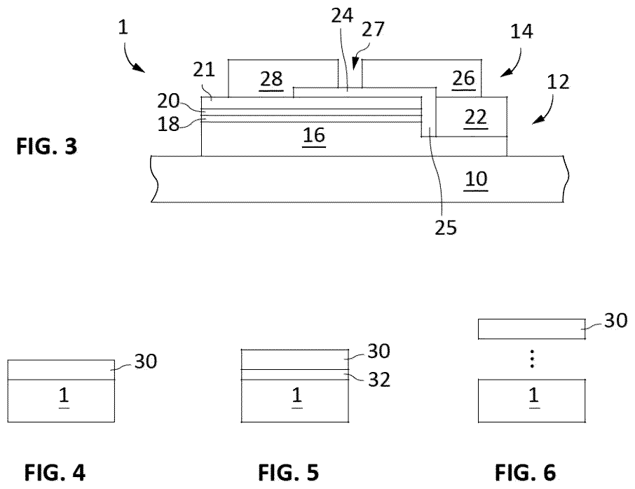

As shown in FIGS. 1 and 2, an illumination device may include a wavelength converting structure that may be used, for example, with light source 100, 200. Light source 100, 200 may be a light emitting diode (LED). Light emitted by the light emitting diode is absorbed by the phosphors in the wavelength converting structure according to embodiments and emitted at a different wavelength. FIG. 3 illustrates one example of a suitable light emitting diode, a III-nitride LED that emits blue light for use in an illumination device such as those disclosed with respect to FIG. 2, in which the SWIR phosphor is combined with a second phosphor that absorbs the blue light and emits the SWIR light.

Though in the example below the semiconductor light emitting device is a III-nitride LED that emits blue or UV light, semiconductor light emitting devices besides LEDs such as laser diodes and semiconductor light emitting devices made from other materials systems such as other III-V materials, III-phosphide, III-arsenide, II-VI materials, ZnO, or Si-based materials may be used, as determined by, for example, the range of wavelengths needed to excite the SWIR phosphor, or combination of SWIR phosphor and second phosphor, in the wavelength converting structure.

FIG. 3 illustrates a III-nitride LED 1 that may be used in embodiments of the present disclosure. Any suitable semiconductor light emitting device may be used and embodiments of the disclosure are not limited to the device illustrated in FIG. 3. The device of FIG. 3 is formed by growing a III-nitride semiconductor structure on a growth substrate 10 as is known in the art. The growth substrate is often sapphire but may be any suitable substrate such as, for example, SiC, Si, GaN, or a composite substrate. A surface of the growth substrate on which the III-nitride semiconductor structure is grown may be patterned, roughened, or textured before growth, which may improve light extraction from the device. A surface of the growth substrate opposite the growth surface (i.e. the surface through which a majority of light is extracted in a flip chip configuration) may be patterned, roughened or textured before or after growth, which may improve light extraction from the device.

The semiconductor structure includes a light emitting or active region sandwiched between n- and p-type regions. An n-type region 16 may be grown first and may include multiple layers of different compositions and dopant concentration including, for example, preparation layers such as buffer layers or nucleation layers, and/or layers designed to facilitate removal of the growth substrate, which may be n-type or not intentionally doped, and n- or even p-type device layers designed for particular optical, material, or electrical properties desirable for the light emitting region to efficiently emit light. A light emitting or active region 18 is grown over the n-type region. Examples of suitable light emitting regions include a single thick or thin light emitting layer, or a multiple quantum well light emitting region including multiple thin or thick light emitting layers separated by barrier layers. A p-type region 20 may then be grown over the light emitting region. Like the n-type region, the p-type region may include multiple layers of different composition, thickness, and dopant concentration, including layers that are not intentionally doped, or n-type layers.

After growth, a p-contact is formed on the surface of the p-type region. The p-contact 21 often includes multiple conductive layers such as a reflective metal and a guard metal which may prevent or reduce electromigration of the reflective metal. The reflective metal is often silver but any suitable material or materials may be used. After forming the p-contact 21, a portion of the p-contact 21, the p-type region 20, and the active region 18 is removed to expose a portion of the n-type region 16 on which an n-contact 22 is formed. The n- and p-contacts 22 and 21 are electrically isolated from each other by a gap 25 which may be filled with a dielectric such as an oxide of silicon or any other suitable material. Multiple n-contact vias may be formed; the n- and p-contacts 22 and 21 are not limited to the arrangement illustrated in FIG. 3. The n- and p-contacts may be redistributed to form bond pads with a dielectric/metal stack, as is known in the art.

In order to form electrical connections to the LED 1, one or more interconnects 26 and 28 are formed on or electrically connected to the n- and p-contacts 22 and 21. Interconnect 26 is electrically connected to n-contact 22 in FIG. 3. Interconnect 28 is electrically connected to p-contact 21. Interconnects 26 and 28 are electrically isolated from the n- and p-contacts 22 and 21 and from each other by dielectric layer 24 and gap 27. Interconnects 26 and 28 may be, for example, solder, stud bumps, gold layers, or any other suitable structure.

The substrate 10 may be thinned or entirely removed. In some embodiments, the surface of substrate 10 exposed by thinning is patterned, textured, or roughened to improve light extraction.

Any suitable light emitting device may be used in light sources according to embodiments of the disclosure. The invention is not limited to the particular LED illustrated in FIG. 3. The light source, such as, for example, the LED illustrated in FIG. 3, is illustrated in the following FIGS. 4, 5 and 6 by block 1.

FIGS. 4, 5, and 6 illustrate devices that combine an LED 1 and a wavelength converting structure 30. The wavelength converting structure 30 may be, for example, wavelength converting structure 108 including an SWIR phosphor as shown in FIG. 1, or wavelength converting structure 218 having an SWIR phosphor and a second phosphor as shown in FIG. 2, according to the embodiments and examples described above.

In FIG. 4, the wavelength converting structure 30 is directly connected to the LED 1. For example, the wavelength converting structure may be directly connected to the substrate 10 illustrated in FIG. 3, or to the semiconductor structure, if the substrate 10 is removed.

In FIG. 5, the wavelength converting structure 30 is disposed in close proximity to LED 1, but not directly connected to the LED 1. For example, the wavelength converting structure 30 may be separated from LED 1 by an adhesive layer 32, a small air gap, or any other suitable structure. The spacing between LED 1 and the wavelength converting structure 30 may be, for example, less than 500 μm in some embodiments.

In FIG. 6, the wavelength converting structure 30 is spaced apart from LED 1. The spacing between LED 1 and the wavelength converting structure 30 may be, for example, on the order of millimeters in some embodiments. Such a device may be referred to as a “remote phosphor” device.

The wavelength converting structure 30 may be square, rectangular, polygonal, hexagonal, circular, or any other suitable shape. The wavelength converting structure may be the same size as LED 1, larger than LED 1, or smaller than LED 1.

In some embodiments, other materials may be added to the wavelength converting structure or the device, such as, for example, materials that improve optical performance, materials that encourage scattering, and/or materials that improve thermal performance.

### Examples

Example 1) Synthesis of Li_{0.532}Ga_{2.35}Sc_{0.104}:Ni_{0.051}. Compositions of Li_{0.532}Ga_{2.35}Sc_{0.1}O₄:Ni_{0.051} were synthesized from powders of lithium carbonate (Alfa Aesar, 99.998%), gallium oxide (Molycorp, UHP grade), scandium oxide (MRE Ltd., 5N) and nickel oxide (Alfa Aesar, 99.99%). 1.595 g of the lithium carbonate, 17.914 g of the gallium oxide, 0.567 g of the scandium oxide, and 0.301 g of the nickel oxide were mixed by ball milling and fired twice at 1300° C. with intermediate milling followed by ball milling, powder washing in water, and final screening.

The obtained powder of Li_{0.532}Ga_{2.35}Sc_{0.104}:Ni_{0.051} crystallized in the inverse spinel structure with a cubic lattice constant of 8.223 Å and a density of 5.812 g/cm³.

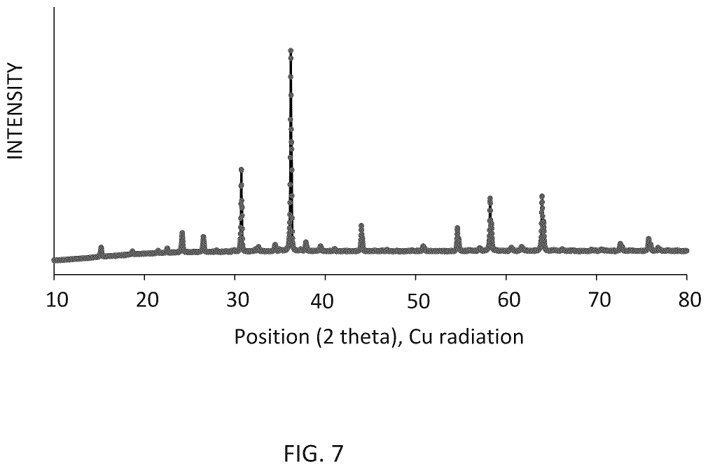

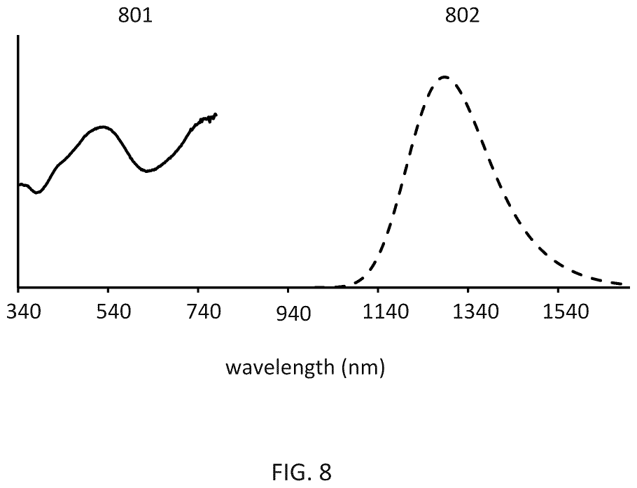

FIG. 7 shows the X ray powder pattern (Cu k alpha radiation) of the obtained Li_{0.532}Ga_{2.35}Sc_{0.104}:Ni_{0.0}si. FIG. 8 shows the powder reflectance 801 and emission (620 nm excitation) spectra 802 of the obtained Li_{0.532}Ga_{2.35}Sc_{0.104}:Ni_{0.051}.

Example 2) Synthesis of Li_{0.536}Al_{1.01}Ga_{1.44}O₄:Ni_{0.051}. Compositions of Li_{0.536}Al_{1.01}Ga_{1.44}O₄:Ni_{0.051} were synthesized from powders of lithium carbonate (Alfa Aesar, 99.998%), gallium oxide (Molycorp, UHP grade), aluminum oxide (Baikowski, 4N) and nickel oxide (Alfa Aesar, 99.99%). 1.968 g of the lithium carbonate, 13.42 g of the gallium oxide, 5.123 g of the aluminum oxide, and 0.375 g of the nickel oxide were mixed by ball milling and fired twice at 1325° C. with intermediate milling followed by ball milling, powder washing in water, and final screening.

The obtained Li_{0.536}Al_{1.01}Ga_{1.44}O₄:Ni_{0.051} powder crystallized in the inverse spinel structure with a cubic lattice constant of 8.086 Å and a density of 5.001 g/cm³.

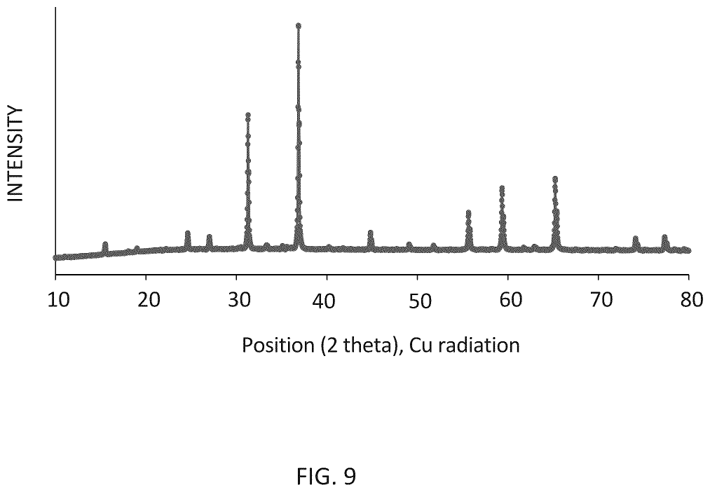

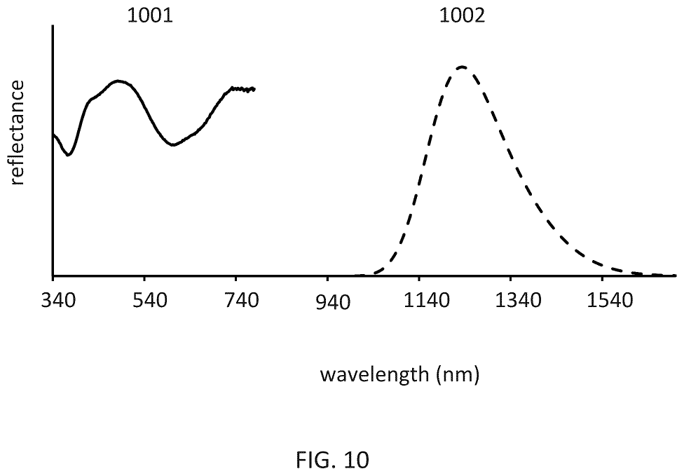

FIG. 9 shows the X ray powder pattern (Cu k alpha radiation) of the obtained Li_{0.536}Al_{1.01}Ga_{1.44}O₄:Ni_{0.051.}. FIG. 10 shows the powder reflectance 1001 and emission (620 nm excitation) spectra 1002 of the obtained Li_{0.536}Al_{1.01}Ga_{1.44}O₄:Ni_{0.051}.

Example 3) Synthesis of Li_{0.273}Mg_{0.5}Sc_{0.05}Ga_{2.16}O₄:Ni_{0.05}. Compositions of Li_{0.273}Mg_{0.5}Sc_{0.05}Ga_{2.1604}:Ni_{0.05} were synthesized from powders of lithium carbonate (Merck, p.a.), magnesium oxide (Merck, p.a.), gallium oxide (Molycorp, UHP grade), scandium oxide (MRE Ltd., 5N), and nickel oxide (Alfa Aesar, 99.99%). 0.935 g of the lithium carbonate, 1.854 g of the magnesium oxide, 18.755 g of the gallium oxide, 0.317 g of the scandium oxide, and 0.344 g of the nickel oxide were mixed by ball milling and fired twice at 1100° C. and 1350° C., respectively, with intermediate milling followed by ball milling, powder washing in water, and final screening.

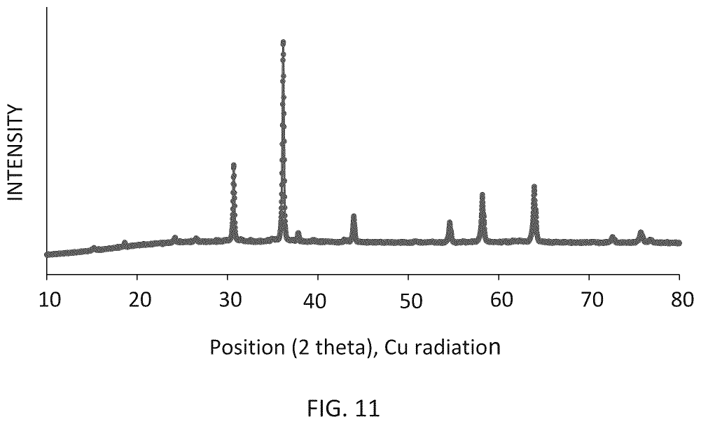

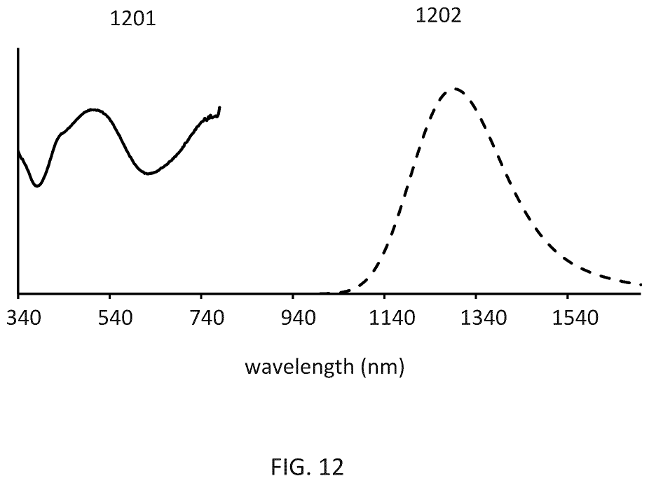

The obtained Li_{0.273}Mg_{0.5}Sc_{0.05}Ga_{2.1604}:Ni_{0.05} powder crystallized in the inverse spinel structure with a cubic lattice constant of 8.2312 Å and a density of 5.178 g/cm³. FIG. 11 shows the X ray powder pattern (Cu k alpha radiation) of the obtained Li_{0.273}Mg_{0.5}Sc_{0.05}Ga_{2.1604}:Ni_{0.05}. FIG. 12 shows the powder reflectance 1201 and emission (620 nm excitation) spectra 1202 of the obtained Li_{0.273}Mg_{0.5}Sc_{0.05}Ga_{2.1604}:Ni_{0.05}.

Example 4) Synthesis of Li_{0.49}Sc_{0.05}Ga_{2.384}O₄:Ni_{0.013},Cr_{0.05}. Compositions of Li_{0.49}Sc_{0.05}Ga_{2.384}O₄:Ni_{0.013},Cr_{0.05} were synthesized from powders of lithium carbonate (Merck p.a.), gallium oxide (Molycorp UHP), nickel oxide (Sigma Aldrich, 99%), scandium oxide (MRE Ltd., 5N), lithium tetraborate (Alfa Aesar, 98%), and chromium oxide (Alfa Aeasar, >98%). 1.517 g of the lithium carbonate, 18.654 g of the gallium oxide, 0.078 g of the nickel oxide, 0.287 g of the scandium oxide, 0.176 g of the lithium tetraborate, and 0.316 g of the chromium oxide were mixed by ball milling and fired at 1300° C. for 4 hrs under ambient conditions. The raw product was milled, washed with diluted hydrochloric acid and water. After screening a powder sample was obtained that crystallized in the cubic spinel structure type with a lattice constant of 8.2127 Å and a density of 5.769 g/cm³.

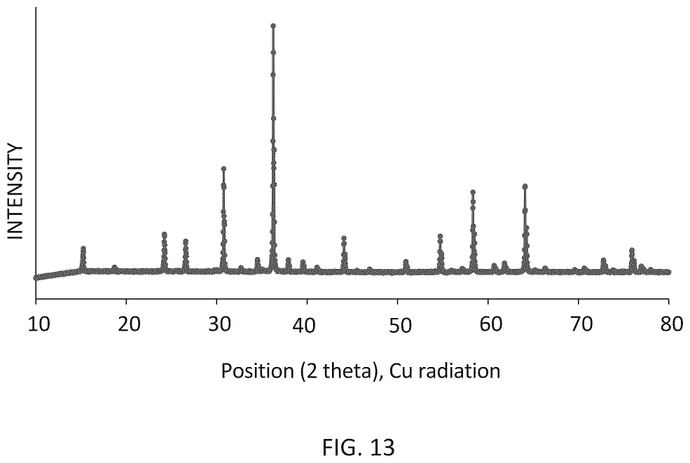

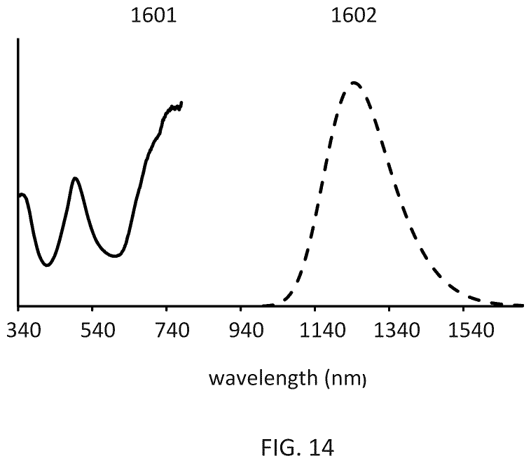

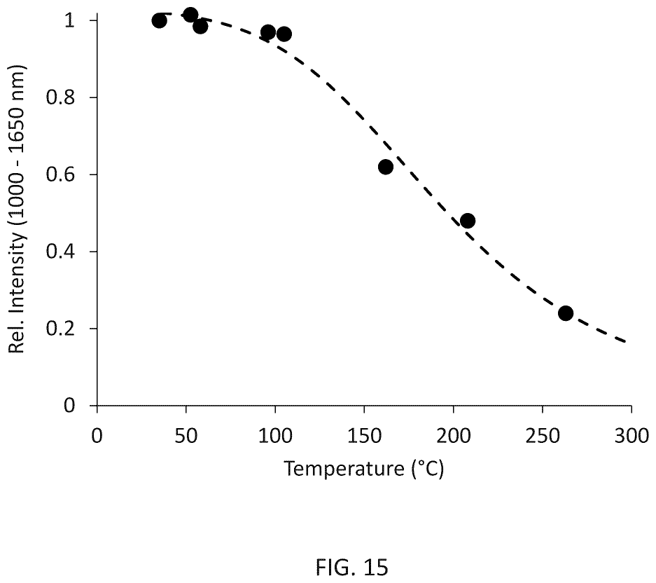

FIG. 13 shows the X ray powder pattern (Cu k alpha radiation) of the obtained Li_{0.49}Sc_{0.05}Ga_{2.384}O₄:Ni_{0.013},Cr_{0.05}. FIG. 14 shows the powder reflectance spectrum 1601 and emission spectrum (for 620 nm excitation) 1602, and FIG. 15 shows the thermal quenching behavior of emission under 620 nm excitation of the obtained Li_{0.49}Sc_{0.05}Ga_{2.384}O₄:Ni_{0.013},Cr_{0.05}.

Example 5) Synthesis of Li_{0.49}Al_{0.05}Ga_{2.384}O₄:Ni_{0.013},Cr_{0.05}. Compositions of Li_{0.49}Al_{0.05}Ga_{2.384}O₄:Ni_{0.013},Cr_{0.05} were synthesized from powders of lithium carbonate (Merck p.a.), gallium oxide (Molycorp UHP), nickel oxide (Sigma Aldrich, 99%), aluminum oxide (Baikowski, >99%), lithium tetraborate (Alfa Aesar, 98%), and chromium oxide (Alfa Aeasar, >98%). 1.522 g of the lithium carbonate, 18.724 g of the gallium oxide, 0.078 g of the nickel oxide, 0.213 g of the aluminum oxide, 0.176 g of the lithium tetraborate, and 0.317 g of the chromium oxide were mixed by ball milling and fired at 1320° C. for 4 hrs under ambient conditions. The raw product was milled, washed with diluted hydrochloric acid and water. After screening a powder sample was obtained that crystallized in the cubic spinel structure type with a lattice constant of 8.1975 Å and a density of 5.779 g/cm³.

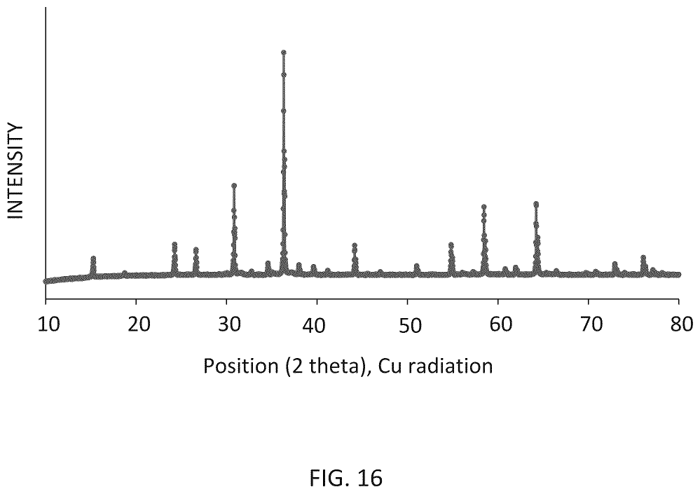

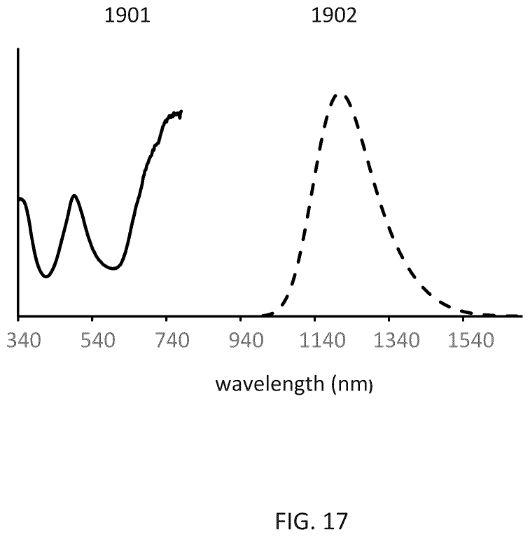

FIG. 16 shows the X ray powder pattern (Cu k alpha radiation) of the obtained Li_{0.49}Al_{0.05}Ga_{2.384}O₄:Ni_{0.013},Cr_{0.05}. FIG. 17 shows the powder reflectance spectrum 1901 and emission spectrum 1902 (for 620 nm excitation) of the obtained Li_{0.49}Al_{0.05}Ga_{2.384}O₄:Ni_{0.013},Cr_{0.05}.

Example 6) Phosphor Converted LED with Red Emitting Pump LED. The Li_{0.49}Sc_{0.05}Ga_{2.384}O₄:Ni_{0.013},Cr_{0.05} spinel type phosphor synthesized as described in Example 5 was mixed with a PDMS type silicone having a refractive index n_{d}=1.41 and a viscosity of 4 Pa s and with a loading of 220 wt % (i.e. 220 g phosphor in 100 g silicone, 69 wt % phosphor in the silicon) with respect to the silicone weight. The suspension was then dispensed into a 2.7×2.0 mm² lead frame LED package equipped with a 622 nm emitting LED die that emits a radiation power of 195 mW at 350 mA and room temperature.

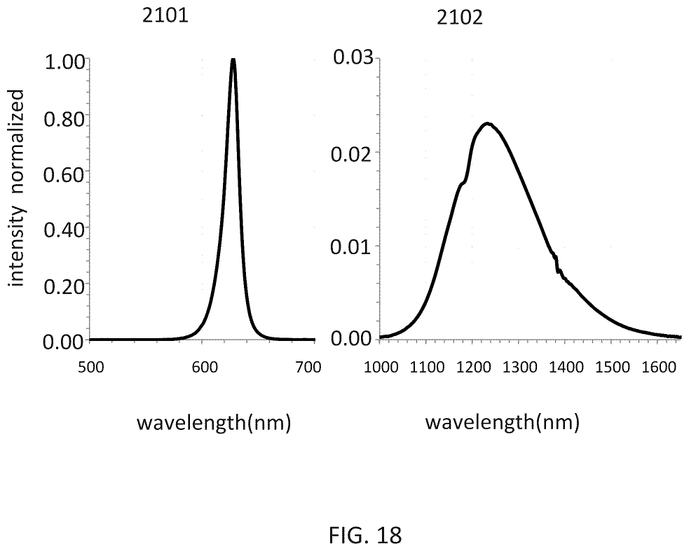

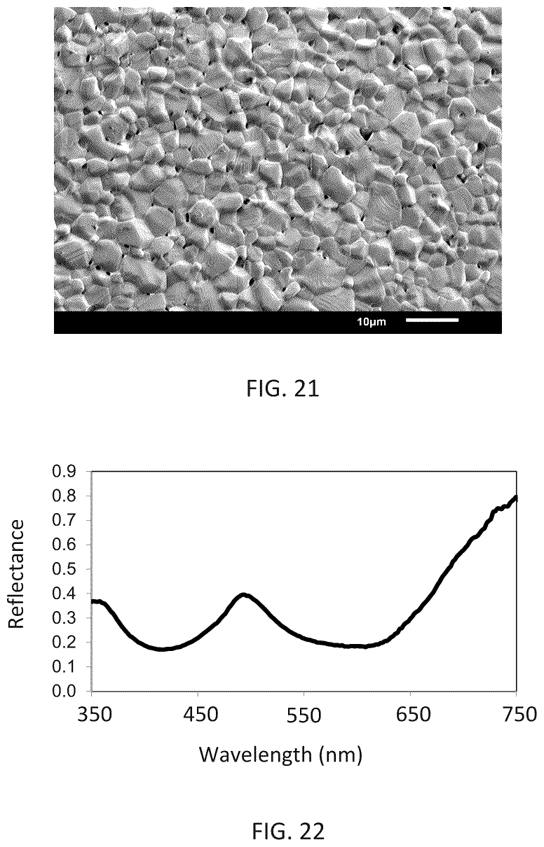

FIG. 18 shows the emission spectrum of the phosphor converted LED of this Example 8 for the visible 2101 and IR 2102 spectral range. The phosphor converted LED shows the same emission band as shown in FIG. 16, except for a small dip in the emission profile at around 1190 nm due to absorption of the silicone encapsulant used. The total emitted IR radiation power in the 1000-1600 nm spectral range is 50-60 mW (power conversion red/IR=25-30% (FIG. 21).

Example 7) Phosphor Converted LED with Blue Emitting InGaN Pump LED.

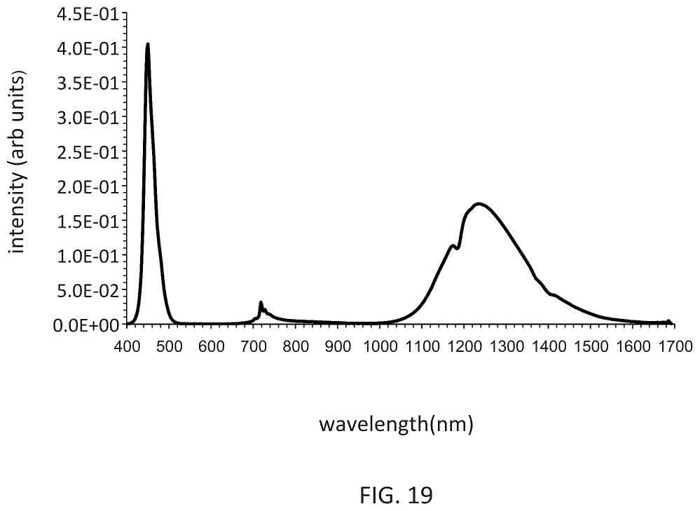

The Li_{0.49}Sc_{0.05}Ga_{2.384}O₄:Ni_{0.013},Cr_{0.05} spinel type phosphor synthesized as described in Example 5 was mixed with a polydimethyl siloxane type silicone encapsulant (weight ratio phosphor/silicone=0.45) and dispensed into 2720 type leadframe LED packages of a size of 2.7 mm×2.0 mm×0.6 mm (L x W x H) equipped with 450 nm emitting InGaN dies (550 mW output power at 350 mA at 25° C. if filled with the same silicone as used for Example 8 above). FIG. 19 shows the total emission spectrum of the pcLED of this Example 9 for 350 mA drive current. In the spectral range of 1000-1700 nm, the LED shows an infrared output power of 171 mW.

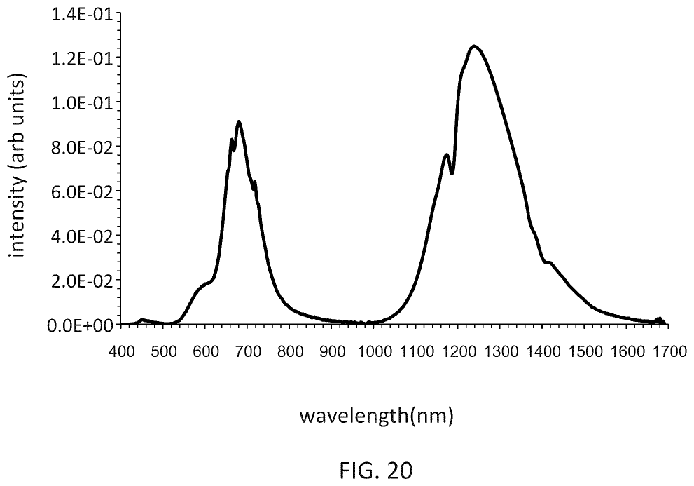

Example 8) Phosphor Converted LED with Blue Emitting InGaN Pump LED and Additional Orange-Red Emitting (Ba_{0.4}Sr_{0.6})_{1.98}Si₅N₈:Eu_{0.02} phosphor. The Li_{0.49}Sc_{0.05}Ga_{2.384}O₄:Ni_{0.013},Cr_{0.05} phosphor synthesized as described in Example 5 was mixed with (Ba_{0.4}Sr_{0.6})_{1.98}Si₅N₈:Eu_{0.02} phosphor and with a polydimethyl siloxane type silicone encapsulant (weight ratio phosphor/silicone=0.45, weight ratio IR phosphor/orange-red phosphor=3) and dispensed into 2720 type leadframe LED packages of a size of 2.7 mm×2.0 mm×0.6 mm (L x W x H) equipped with 450 nm emitting InGaN dies (550 mW output power at 350 mA at 25° C. if filled with the same silicone as used for example 8 above). [00105](Ba_{0.4}Sr_{0.6})_{1.98}Si₅N₈:Eu_{0.02} phosphor was made by mixing BaH2 (Materion, >99%), SrH2 (Materion, >99%), Eu2O3 (Neo, >99.9%) and Si3N4 (UBE, >98%) under inert conditions and firing the mixture twice with milling in-between the firings under forming gas at 1680° C. The phosphor is then washed in diluted mineral acid, water and ethanol followed by ball milling. FIG. 20 shows the total emission spectrum of Example 10 for 350 mA drive current. In the spectral range 1000-1700 nm the LED shows an infrared output power of 139 mW.

Example 9) Synthesis of Li_{0.49}Al_{0.05}Ga_{2.384}O₄:Ni_{0.013},Cr_{0.05} polycrystalline ceramic. 11.11 g Lithium carbonate (Merck, p.a.), 136.96 g Gallium oxide (Dowa Electronics, 4N), 0.569 g Nickel oxide (Sigma Aldrich, 99%), 1.542 g aluminum oxide (Baikowski, SP-DBM), 2.315 g chromium oxide (Materion, 3N5) and 2.67 g aluminum stearate (Alfa Aesar, techn. grade) are mixed by planetary ball milling and fired at 850° C. for 4 hrs. After firing, the precursor powder is ball milled with alumina milling media in ethanol until an average particle size in the 0.6-0.7 μm range is being obtained, cast on a mylar film with a polyvinyl butyral binder system, and dried. After peel-off of the ceramic tapes, a forming procedure including cutting, stacking, lamination and binder burn out has been applied followed by firing of the ceramic green bodies in air atmosphere at 1350° C. for 8 hrs.

The obtained ceramic wafers show a polycrystalline structure with grains crystallizing in the cubic spinel structure with a lattice constant a₀=8.1976 Å as measured by X ray diffraction and a density of 97% (relative to the theoretical density calculated from the lattice constants). FIG. 21 shows the morphology of the as-sintered ceramic (top surface). FIG. 22 shows the reflectance spectrum of the as-sintered ceramic.

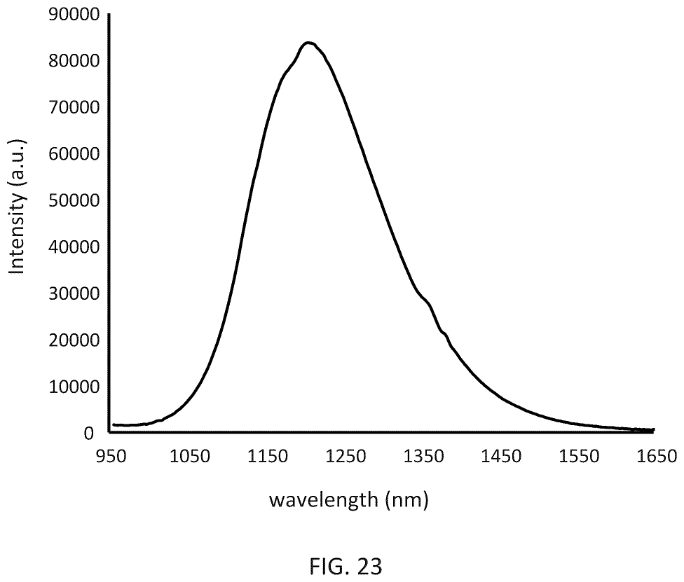

Example 10) pcLED comprising the ceramic converter element of Example 9. After dicing the ceramic wafers of Example 9 into platelets of 1×1 mm lateral size and thicknesses in the 150-200 μm range the platelets were attached on top of InGaN primary LED light sources by means of a thermally curable silicone resin. FIG. 23 shows the emission spectrum of the pcLED with a peak wavelength at 1200 nm and a spectral width (FWHM) of 186 nm which allows axial resolutions in the 3-4 μm range in OCT applications.

This disclosure is illustrative and not limiting. Further modifications will be apparent to one skilled in the art in light of this disclosure and are intended to fall within the scope of the appended claims.

1. A light source for an OCT system, the light source comprising:
a light emitting diode; and
a wavelength converting structure comprising a spinel-type luminescent material having peak emission in the range 1100-1300 nm with a FWHM in the range 170-240 nm.

2. The light source of claim 1, wherein the spinel-type luminescent material has peak emission in the range 1200-1250 nm.

3. The light source of claim 1, wherein the spinel-type luminescent material has the formula AE_{1-x-z}A_{z+0.5(x-y)}D_{2+0.5(x-y)-z-u}E_{z}O₄:Ni_{y},Crᵤ; where
AE=Mg, Zn, Co, Be or mixtures thereof;
A=Li, Na, Cu, Ag or mixtures thereof;
D=Ga, Al, B, In, Sc or mixtures thereof;
E=Si, Ge, Sn, Ti, Zr, Hf or mixtures thereof; and
0≤x≤1, 0<y≤0.1, 0≤z≤1, 0≤u≤0.2.

4. The light source of claim 3, wherein the spinel-type luminescent material has the formula Li_{0.5-0.5y}D_{2.5-0.5y-u}O₄:Ni_{y},Crᵤ with 0<y≤0.1, 0≤u≤0.2.

5. The light source of claim 4, wherein the spinel-type luminescent material has the formula Li_{0.49}Al_{0.05}Ga_{2.384}O₄:Ni_{0.013},Cr_{0.05}.

6. The light source of claim 1, wherein the wavelength converting structure comprises the spinel-type luminescent material dispersed in a binder.

7. The light source of claim 6, wherein the binder comprises a methyl polysiloxane resin.

8. The light source of claim 6, wherein the binder comprises a dimethyl polysiloxane resin.

9. The light source of claim 1, wherein the wavelength converting structure comprises the spinel-type luminescent material in ceramic form.

10. The light source of claim 1, wherein the light emitting diode is an InGaN light emitting diode configured to emit light in the 420-450 nm range and the spinel-type luminescent material is arranged to absorb the blue light and in response emit with the peak emission in the range 1100-1300 nm with a FWHM in the range 170-240 nm.

11. The light source of claim 10, wherein the wavelength converting structure comprises the spinel-type luminescent material in ceramic form.

12. The light source of claim 11 wherein the spinel-type luminescent material has the formula AE_{1-x-z}A_{z+0.5(x-y)}D_{2+0.5(x-y)-z-u}E_{z}O₄:Ni_{y},Crᵤ; where
AE=Mg, Zn, Co, Be or mixtures thereof;
A=Li, Na, Cu, Ag or mixtures thereof;
D=Ga, Al, B, In, Sc or mixtures thereof;
E=Si, Ge, Sn, Ti, Zr, Hf or mixtures thereof; and
0≤x≤1, 0<y≤0.1, 0≤z≤1, 0≤u≤0.2.

13. The light source of claim 12, wherein the spinel-type luminescent material has the formula Li_{0.5-0.5y}D_{2.5-0.5y-u}O₄:Ni_{y},Crᵤ with 0<y≤0.1, 0≤u≤0.2.

14. The light source of claim 13 wherein the spinel-type luminescent material has the formula Li_{0.49}Al_{0.05}Ga_{2.384}O₄:Ni_{0.013},Cr_{0.05}.

15. The light source of claim 14, wherein the spinel-type luminescent material has peak emission in the range 1200-1250 nm.

16. An OCT system comprising:
the light source of claim 1;
a sample arm;
a reference arm; and
a detector arranged to produce an interference signal from light emitted by the light source and coupled into the sample arm and light emitted from the light source and coupled into the reference arm.

17. The OCT system of claim 16, wherein the light source provides an axial resolution of less than 4 microns.
